(19) United States
(12) Patent Application Publication
(10) Pub. No.: US 2010/0021977 A1
(43) Pub. Date: Jan. 28, 2010

(54) PROCESS FOR PREPARING ALKYL (METH)ACRYLATES USING AN ENZYMATIC CYANOHYDRIN HYDROLYSIS

(75) Inventors: Alexander May, Darmstadt (DE); Jochen Ackermann, Muehltal (DE); Hermann Siegert, Seeheim-Jugenheim (DE); Bernd Vogel, Wiesbaden (DE); Steffen Osswald, Rodenbach (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: Evonik Roehm GmbH, Darmstadt (DE)

(21) Appl. No.: 12/441,145

(22) PCT Filed: Aug. 30, 2007
(86) PCT No.: PCT/EP2007/059033

§ 371 Date: Mar. 13, 2009

(30) Foreign Application Priority Data
Nov. 22, 2006 (DE) 102006055426.4

(51) Int. Cl. C12P 7/62

(52) U.S. Cl. 435/135

(57) ABSTRACT

The present invention relates to a process for preparing alkyl(meth)acrylates, characterized in that the process has a step in which a cyanohydrin is hydrolysed with an enzyme whose residual activity after the conversion of methacrylonitrile in the presence of 20 mM cyanide ions at 20° C. after 30 min is at least 90% of the residual activity of the enzyme which has been used under otherwise identical conditions in the absence of cyanide ions.

The present invention relates to processes for preparing alkyl(meth)acrylates using an enzymatic cyanohydrin hydrolysis.

Acrylic esters and methacrylic esters, referred to hereinafter as alkyl(meth)acrylates, find their main field of use in the preparation of polymers and copolymers with other polymerizable compounds.

Methacrylic ester, for example methyl methacrylate, is additionally an important starting material for various specialty esters which are based on methacrylic acid (MAA) and are prepared by transesterification with the corresponding alcohol.

Methyl methacrylate (MMA) and methacrylic acid are nowadays prepared predominantly starting from hydrocyanic acid and acetone via the acetone cyanohydrin (ACH) which forms as a central intermediate.

Further processes which use a raw material basis other than ACH have been described in the relevant patent literature and some of them have been realized on the production scale. In this context, C-4 based raw materials such as isobutylene or tert-butanol are used nowadays as reactants, which are converted to the desired methacrylic acid derivatives via several process stages.

There has additionally been intensive investigation of the use of propene as a base raw material, which affords methacrylic acid in moderate yields via the stages of hydrocarbonylation (to isobutyric acid) and dehydrogenating oxidation.

It is known than propanal or propionic acid, which are obtainable in industrial processes starting from ethylene and C-1 units such as carbon monoxide, can be used as a base raw material. In these processes, reaction is effected with formaldehyde in an aldolizing reaction with dehydration of the β-hydroxycarbonyl compound formed in situ to give the corresponding α,β-unsaturated compound. An overview of the common processes for preparing methacrylic acid and its esters can be found in the literature, such as Weissermel, Arpe “Industrielle organische Chemie”, VCH, Weinheim 1994, 4th edition, p. 305 ff., or Kirk Othmer “Encyclopedia of Chemical Technology”, 3rd edition, Vol. 15, page 357.

It is common knowledge that technical processes based on ACH can be performed with highly concentrated sulphuric acid (around about 100% by weight of H₂SO₄) in the first step of the reaction, the so-called amidation, at temperatures between 80° C. and about 110° C.

A representative example of such a process is U.S. Pat. No. 4,529,816, in which the ACH amidation is performed at temperatures around 100° C. with a molar ratio of ACH:H₂SO₄ of about 1:1.5 to 1:1.8. Process steps relevant to this process are: a) amidation; b) conversion; and c) esterification.

In addition to the poor overall yields of the above-described process, which, especially on the production scale, are associated with the occurrence of considerable amounts of wastes and offgases, this process has the disadvantage that far greater than stoichiometric amounts of sulphuric acid have to be used. In addition, tar-like, solid condensation products which prevent trouble-free conveying of the process acid and have to be removed with considerable difficulty separate out of the ammonium hydrogensulphate- and sulphuric acid-containing process acid which is regenerated in a sulphuric acid contact plant.

Owing to the drastic yield losses in the above-described process from U.S. Pat. No. 4,529,816, there are some proposals to amidate and hydrolyse ACH in the presence of water, the hydroxyl function in the molecule being retained at least in the first steps of the reaction.

Depending on whether they are performed in the presence of or without methanol, these proposals for an alternative amidation in the presence of water lead either to the formation of methyl 2-hydroxyisobutyrate (=MHIB) or to the formation of 2-hydroxyisobutyric acid (=HIBA).

2-Hydroxyisobutyric acid is a central intermediate for the preparation of methacrylic acid and methacrylic esters derived therefrom, especially methyl methacrylate.

A further alternative for the preparation of esters of 2-hydroxyisobutyric acid, especially methyl 2-hydroxyisobutyrate, proceeding from ACH is described in JP Hei-4-193845. In JP Hei-4-193845, ACH is first amidated with from 0.8 to 1.25 equivalents of sulphuric acid in the presence of less than 0.8 equivalent of water below 60° C. and then reacted at temperatures of greater than 55° C. with more than 1.2 equivalents of alcohol, especially methanol, to give MHIB or corresponding esters. There is no discussion here on the presence of viscosity-reducing media which are stable towards the reaction matrix.

The disadvantages and problems of this process are the industrial implementation as a result of exceptional viscosity formation at the end of the reaction.

Some approaches to the utilization and conversion of MHIB by dehydration to methyl methacrylate are described in the patent literature.

It is also known that 2-hydroxyisobutyric acid can be prepared starting from acetone cyanohydrin (ACH) by performing the hydrolysis of the nitrile function in the presence of mineral acids (see J. Brit. Chem. Soc. (1930); Chem. Ber. 72 (1939), 800).

A representative example of such a process is the Japanese patent publication Sho 63-61932, in which ACH is hydrolysed in a two-stage process to give 2-hydroxyisobutyric acid. In this process, ACH is first converted in the presence of 0.2-1.0 mol of water and 0.5-2 equivalents of sulphuric acid to form the corresponding amide salts. Even in this step, in the case of use of small water and sulphuric acid concentrations which are necessary to obtain good yields, short reaction times and small amounts of waste process acid, massive problems occur with the stirrability of the amidation mixture as a result of high viscosity of the reaction mixtures, especially towards the end of the reaction time.

When the molar amount of water is increased to ensure a low viscosity, the reaction slows drastically and side reactions occur, especially the fragmentation of ACH to the acetone and hydrocyanic acid reactants, which react further under the reaction conditions to give conversion products. Even when the temperature is increased, according to the specifications of the problem in the Japanese patent publication SHO 63-61932, it is possible to control the viscosity of the reaction mixture and the corresponding reaction mixtures become stirrable as a result of the falling viscosity, but the side reactions here too increase drastically even at moderate temperatures, which is ultimately manifested in only moderate yields (see comparative examples).

When working at low temperatures of <50° C., which would enable a selective reaction, the increase in the concentration of the amide salts which are sparingly soluble under the reaction conditions towards the end of the reaction initially results in formation of a suspension which is difficult to stir and finally in the complete solidification of the reaction mixture.

In the second step of the Japanese patent publication SHO 63-61932, water is added to the amidation solution and hydrolysis is effected at higher temperatures than the amidation temperature to form 2-hydroxyisobutyric acid from the amide salts formed after the amidation with release of ammonium hydrogensulphate.

An essential factor for the economic viability of an industrial process is, in addition to the selective preparation of the HIBA target product in the reaction, also the isolation from the reaction matrix and the removal of HIBA from the remaining process acid.

JP Sho 57-131736, method for isolating alpha-oxyisobutyric acid (=HIBA), addresses these problems by treating the reaction solution which is obtained by hydrolytic cleavage after the reaction between acetone cyanohydrin, sulphuric acid and water and comprises alpha-hydroxyisobutyric acid and acidic ammonium hydrogensulphate with an extractant, which transfers the 2-hydroxyisobutyric acid into the extractant and leaves the acidic ammonium sulphate in the aqueous phase.

In this process, the sulphuric acid which is still free in the reaction medium is neutralized before the extraction by treatment with an alkaline medium, in order to increase the degree of extraction of HIBA into the organic extraction phase. The necessary neutralization is associated with considerable additional use of aminic or mineral base and hence with considerable waste amounts of corresponding salts, which cannot be disposed of in an ecologically and economically viable manner.

The disadvantages of the JP Sho 57-131736 process for preparing MMA via methacrylamide hydrogensulphate (reaction sequence: amidation-conversion-hydrolytic esterification) can be summarized as follows:
a.) Use of high molar sulphuric acid excesses based on ACH (in the industrial process approx. 1.5-2 equivalents of sulphuric acid per equivalent of ACH).
b.) High yield losses in the amidation step (approx. 3-4%) and in the conversion step (approx. 5-6%), which is ultimately manifested in a maximum methacrylamide sulphate yield of approx. 91%.
c.) Large waste streams in the form of aqueous sulphuric acid in which ammonium hydrogensulphate and organic by-products are dissolved. Deposition of undefined tar residues from this process waste acid, which necessitates an aftertreatment or complicated disposal.

The disadvantages of the JP Sho 57-131736 process for preparing MMA via hydroxyisobutyric acid as the central intermediate (reaction sequence: amidation-hydrolysis-HIBA synthesis-MAA synthesis-hydrolytic esterification) can be summarized as follows:
a.) Use of small molar sulphuric acid excesses based on ACH (only approx. 1.0 equivalent of sulphuric acid per equivalent of ACH) but massive problems with viscosity and stirrability of the amidation medium up to complete solidification of the reaction mixtures; the proposed dilution of the amidation with alcohols (methanol) or various esters leads, under the reaction conditions, to incomplete ACH conversion, drastic increase in the side reactions or to chemical decomposition of the diluents.
b.) High yield losses in the amidation step (approx. 5-6%) and complicated extraction with an organic solvent to form an extractant phase comprising water and HIBA, which has to be worked up by distillation with high energetic input to isolate HIBA. Per kg of HIBA, about 2 kg of process acid waste are generated, which contains about 34% by weight of water as well as 66% by weight of ammonium hydrogensulphate (see Japanese publication SHO-57-131736, Example 4). The regeneration of a waste acid solution with high water contents in a sulphuric acid contact plant (=SC plant) is associated with a considerable energy input which significantly limits the capacity of such an SC plant.

A common factor in all of these processes is that the isolation of HIBA from the ammonium hydrogensulphate-containing aqueous reaction matrix is very complicated. Too high a water content in the HIBA-containing extraction phase also causes entrainment of ammonium hydrogensulphate into the subsequent MAA stage, which can no longer be operated on the industrial scale over an acceptable period. The high energy input in the regeneration of highly concentrated aqueous process acid and also extraction streams additionally make the procedures proposed uneconomic and do not offer any real alternative to the established procedure, which may be unselective but is appropriate to the purpose owing to the few simple process operations.

EP 0 487 853 describes the preparation of methacrylic acid starting from acetone cyanohydrin (ACH), characterized in that, in the first step, ACH is reacted with water at moderate temperatures in the presence of a heterogeneous hydrolysis catalyst and, in the second step, 2-hydroxyisobutyramide is reacted with methyl formate or methanol/carbon monoxide to form formamide and methyl hydroxyisobutyrate, and, in the third step, MHIB is hydrolysed in the presence of a heterogeneous ion exchanger with water to give hydroxyisobutyric acid, and, in the fourth step, HIBA is dehydrated by allowing it to react in the liquid phase at high temperatures in the presence of a soluble alkali metal salt. Methacrylic acid preparation ex HIBA is described at high conversions around 99% with more or less quantitative selectivities. The multitude of reaction steps needed and the necessity of intermediate isolation of individual intermediates, especially including the performance of individual process steps at elevated pressure, make the process complicated and hence ultimately uneconomic.

Some approaches to the utilization and conversion of MHIB by dehydration to give methyl methacrylate have been described in the patent literature.

For example, in EP 0 429 800, MHIB or a mixture of MHIB and of a corresponding alpha- or beta-alkoxy ester in the gas phase, in the presence of methanol as a co-feed, is converted over a heterogeneous catalyst consisting of a crystalline aluminosilicate and a mixed dopant composed firstly of an alkali metal element and secondly of a noble metal.

A similar approach is followed by EP 0 941 984, in which the gas phase dehydration of MHIB is described as a sub-step of an MMA synthesis in the presence of a heterogeneous catalyst consisting of an alkali metal salt of phosphoric acid on SiO₂. However, this multistage process is complicated overall, requires elevated pressures and hence expensive equipment in some steps, and affords only unsatisfactory yields.

A central step in the preparation of alkyl(meth)acrylates according to the processes of documents EP 0 429 800, EP 0 487 853 and EP 0 941 984 is the hydrolysis of the cyanohydrin to the carboxamide. In general, catalysts comprising manganese dioxide can be used for this purpose. By way of example for many documents, reference is made to the publication DE 1593320. DE 1593320 describes a process for hydrolysing nitriles to amides with the aid of manganese dioxide, in which yields up to over 90% have been achieved with aliphatic nitrites. This process affords good yields with a high rate. However, a disadvantage is the low lifetime of the catalyst. In continuous processes, production therefore has to be interrupted after a short time to exchange the catalyst. This operation is associated with very high costs. Although many efforts have been undertaken to improve it, the limited lifetime of the catalyst constitutes a high cost factor in the production of alkyl(meth)acrylates in the processes detailed above.

In addition, the use of enzymes to prepare carboxamides from cyanohydrins is known. The suitable enzymes include nitrile hydratases. This reaction is described by way of example in “Screening, Characterization and Application of Cyanide-resistant Nitrile Hydratases” Eng. Life. Sci. 2004, 4, No. 6. However, the productivity of this reaction is very low, so that this way of preparing the carboxamides has to date not gained any industrial significance for the production of alkyl(meth)acrylates.

In view of the prior art, it is thus an object of the present invention to provide processes for preparing alkyl(meth)acrylates which can be performed in a particularly simple and inexpensive manner and with high yield. A particular problem consists in particular in providing a process which ensures a particularly long lifetime of the catalysts used with high rate, low energy input and low yield losses.

This object and further objects which are not stated explicitly but which can be derived or discerned immediately from the connections discussed herein by way of introduction are achieved by a process having all features of claim 1. Appropriate modifications to the processes according to the invention are protected in subclaims.

By virtue of a process for preparing alkyl(meth)acrylates having a step in which a cyanohydrin is hydrolysed with an enzyme whose residual activity after the conversion of methacrylonitrile in the presence of 20 mM cyanide ions at 20° C. after 30 min is at least 90% of the residual activity of the enzyme which has been used under otherwise identical conditions in the absence of cyanide ions, it is possible to provide a process for preparing alkyl(meth)acrylates which can be performed in a particularly simple and inexpensive manner and with high yield.

At the same time, the processes according to the invention can achieve a series of further advantages. One is that the lifetime of the catalyst can surprisingly be greatly prolonged by the process according to the invention. This allows the process to be performed particularly efficiently and inexpensively, since operational shutdown to change the catalyst is necessary only rarely in the case of continuous operation of the plant. In addition, the catalyst to be used in accordance with the invention can be obtained in a very simple and inexpensive manner. In addition, preferred enzymes which can be used for the hydrolysis of the cyanohydrin exhibit surprisingly high productivity.

The process avoids the use of sulphuric acid in high amounts as a reactant. Accordingly, large amounts of ammonium hydrogensulphate are not obtained in the process according to the invention.

In this context, the formation of by-products is unusually low. In addition, especially taking account of the high selectivity, high conversions are achieved. The process of the present invention has a low degree of formation of by-products.

The process according to the invention enables the efficient preparation of alkyl(meth)acrylates. Alkyl(meth)acrylates are esters which are derived from the (meth)acrylic acids. The term (meth)acrylic acid refers to methacrylic acid, acrylic acid and mixtures of the two. In addition to acrylic acid (propenoic acid) and methacrylic acid (2-methyl-propenoic acid), they include in particular derivatives which comprise substituents. The suitable substituents include in particular halogens, such as chlorine, fluorine and bromine, and alkyl groups which may comprise preferably 1 to 10, more preferably 1 to 4, carbon atoms. They include β-methylacrylic acid (butenoic acid), α,β-dimethylacrylic acid, β-ethylacrylic acid, and β,β-dimethylacrylic acid. Preference is given to acrylic acid (propenoic acid) and methacrylic acid (2-methylpropenoic acid), particular preference being given to methacrylic acid. The alcohol radical of preferred alkyl(meth)acrylates comprises preferably 1 to 20 carbon atoms, in particular 1 to 10 carbon atoms and more preferably 1 to 5 carbon atoms. Preferred alcohol radicals derive in particular from methanol, ethanol, propanol, butanol, in particular n-butanol and 2-methyl-1-propanol, pentanol, hexanol and 2-ethylhexanol, particular preference being given to methanol and ethanol. The preferred alkyl(meth)acrylates include in particular methyl methacrylate, methyl acrylate, ethyl methacrylate and ethyl acrylate.

The process according to the invention has a step in which a cyanohydrin is hydrolysed with an enzyme whose residual activity after the conversion of methacrylonitrile in the presence of 20 mM cyanide ions at 20° C. after 30 min is at least 90% of the residual activity of the enzyme which has been used under otherwise identical conditions in the absence of cyanide ions. In a preferred aspect of the present invention, the residual activity after the conversion in the presence of 50 mM cyanide ions may be at least 60%.

The form in which the enzyme is used is generally uncritical. For example, the enzyme may be used in the form of microorganisms which comprise the enzyme. In this context, it is also possible to use a lysate of these microorganisms. For this purpose, preference is given to using microorganisms which produce the enzyme. In a particular aspect of the present invention, resting cells of these microorganisms may be used. In this context, it is possible to use natural microorganisms or microorganisms which have been isolated and purified. “Isolated and purified microorganisms” relates to microorganisms which are present in a higher concentration than is to be found in nature. Moreover, the enzyme, which can be referred to as a nitrile hydratase, may also be used in purified form.

In a preferred embodiment of the present invention, the enzyme may stem from microorganisms of the Pseudomonas genus. The particularly preferred microorganisms of the Pseudomonas genus include Pseudomonas marginalis or Pseudomonas putida. Particularly preferred microorganisms of the Pseudomonas genus from which enzymes usable in accordance with the invention stem have been deposited under the number DSM 16275 and DSM 16276. The deposition was on Sep. 3, 2004 at the DSMZ, Deutsche Sammlung für Mikroorganismen und Zellkulturen [German collection of microorganisms and cell cultures] in Brunswick, under the Budapest Agreement. These strains are particularly suitable for producing the inventive enzymes.

The microorganisms or enzymes can be obtained, for example, by a process in which
a) a microorganism which produces this nitrile hydratase, especially of the Pseudomonas marginalis or Pseudomonas putida genus, is fermented under conditions such that the enzyme forms in the microorganism,
b) the cells are harvested at the earliest after they have passed through the logarithmic growth phase and
c) either the microorganism comprising the enzyme, if appropriate after increasing the permeability of the cell membrane, or
d) the lysate of the cells or
e) the enzyme present in the cells of the microorganism
   can be isolated by known measures. The microorganism can preferably be isolated as a resting cell.

The culture medium to be used should suitably satisfy the requirements of the particular strains. Descriptions of culture media of various microorganisms are present in the handbook “Manual of Methods for General Bacteriology” of the American Society for Bacteriology (Washington D.C., USA, 1981).

The carbon sources used may be sugars and carbohydrates, for example glucose, sucrose, lactose, fructose, maltose, molasses, starch and cellulose, oils and fats, for example soya oil, sunflower oil, groundnut oil and coconut fat, fatty acids, for example palmitic acid, stearic acid and linoleic acid, alcohols, for example glycerol and ethanol, and organic acids, for example acetic acid. These substances may be used individually or as a mixture.

The nitrogen sources used may advantageously be organic nitrites or acid amides such as acetonitrile, acetamide, methacrylonitriles, methacrylamide, isobutyronitrile, isobutyramide or urea, also in combination with other nitrogen compounds such as peptones, yeast extract, meat extract, malt extract, corn steep liquor, soybean flour, and/or inorganic compounds such as ammonium sulphate, ammonium chloride, ammonium phosphate, ammonium carbonate and ammonium nitrate. The nitrogen sources may be used individually or as a mixture.

The phosphorus source used may be phosphoric acid, potassium dihydrogenphosphate or dipotassium hydrogenphosphate, or the corresponding sodium salts.

The culture medium generally further comprises salts of metals, for example magnesium sulphate or iron sulphate, which are necessary for growth. Finally, it is possible to use essential growth substances such as amino acids and vitamins in addition to the abovementioned substances. The feedstocks mentioned may be added to the culture in a single batch or be fed in in a suitable manner during the culture.

The pH of the culture is controlled by using basic compounds such as sodium hydroxide, potassium hydroxide, ammonia or aqueous ammonia, or acidic compounds such as phosphoric acid or sulphuric acid, in a suitable manner.

To control the evolution of foam, antifoams, for example fatty acid polyglycol esters, are used. In order to maintain aerobic conditions, oxygen or oxygen-containing gas mixtures, for example air, are introduced into the culture.

The temperature of the culture is normally 10 to 40° C. and preferably 10 to 30° C. The culture can preferably be continued until it has passed through the logarithmic growth phase. This aim is normally achieved within 10 hours to 70 hours. Thereafter, the cells are preferably harvested, washed and taken up in a buffer as a suspension at a pH of 6-9, in particular of 6.8 to 7.9. The cell concentration is 1-25%, in particular 1.5 to 15% (moist weight/v). The permeability can be increased by physical or chemical methods, for example with toluene as described in Wilms et al., J. Biotechnol., Vol. 86 (2001), 19-30, such that the cyanohydrin to be converted can penetrate through the cell wall and the carboxamide can leave.

According to the invention, cyanohydrins (α-hydroxycarbonitriles) are used. These compounds are known per se and are described, for example, in Römpp Chemie Lexikon, 2nd edition on CD-ROM. The preferred cyanohydrins include hydroxyacetonitrile, 2-hydroxy-4-methylthiobutyronitrile, α-hydroxy-γ-methylthiobutyronitrile (4-methylthio-2-hydroxybutyronitrile), 2-hydroxypropionitrile (lactonitrile) and 2-hydroxy-2-methyl-propionitrile (acetone cyanohydrin), particular preference being given to acetone cyanohydrin.

The concentration of the cyanohydrins to be converted in the reaction solution is not restricted to particular ranges.

In order to prevent inhibition of the enzyme activity by the substrate, the concentration of the cyanohydrin is generally kept at 0.02 to 10 w/w %, in particular 0.1 to 2 w/w %, based on the amount of the biocatalyst as a dried cell mass. The substrate can be added in its entirety at the start of the reaction, or continuously or batchwise in the course of the reaction.

The dry weight is determined with the MA 45 moisture analyser (Sartorius).

The water which is needed to hydrolyse the cyanohydrin can in many cases be used as the solvent.

The water used for the hydrolysis may have a high purity. However, this property is not obligatory. Thus, as well as fresh water, it is also possible to use service water or process water which comprises greater or lesser amounts of impurities. Accordingly, it is also possible to use recycled water for the hydrolysis.

When the solubility of the cyanohydrin in the aqueous reaction system is too low, a solubilizer can be added. The reaction can, though, alternatively also be performed in a biphasic water/organic solvent system.

When cells of the microorganism are used as the enzymatically active material, the amount of the cells used, in relation to the amount of substrate, is preferably 0.02 to 10 w/w % as a dried cell mass.

It is also possible to immobilize the isolated enzyme by commonly known techniques and then to use it in this form.

In addition, further constituents may be present in the reaction mixture for the hydrolysis of the carbonitrile. These include carbonyl compounds such as aldehydes and ketones, especially those which have been used to prepare cyanohydrins to be used with preference as the carbonitrile. For example, acetone and/or acetaldehyde may be present in the reaction mixture. This is described, for example, in U.S. Pat. No. 4,018,829-A. The purity of the aldehydes and/or ketones added is generally not particularly critical. Accordingly, these substances may comprise impurities, especially alcohols, for example methanol, water and/or methyl α-hydroxyisobutyrate (MHIB). The amount of carbonyl compounds, especially acetone and/or acetaldehyde, may be used within wide ranges in the reaction mixture. The carbonyl compound is preferably used in an amount in the range of 0.1-6 mol, preferably 0.1-2 mol, per mole of carbonitrile.

In a particular aspect of the present invention, the hydrolysis of the cyanohydrin can be performed in the presence of hydrocyanic acid or a salt of hydrocyanic acid. In this context, the starting concentration of cyanide is preferably in the range of 0.1 mol % to 3 mol % of cyanide, more preferably 0.5 to 3 mol % of cyanide, based on the cyanohydrin used.

The temperature at which the hydrolysis reaction of the cyanohydrin is effected may generally be within the range of −5 to 50° C., preferably in the range of 0 to 40° C. and more preferably in the range of 10 to 30° C.

Depending on the reaction temperature, the hydrolysis reaction can be performed at reduced or elevated pressure. Preference is given to performing this reaction within a pressure range of 0.1-10 bar, more preferably 0.5-5 bar.

The reaction time of the hydrolysis reaction depends upon factors including the carbonitriles used, the activity of the catalyst and the reaction temperature, and this parameter may be within wide ranges. The reaction time of the hydrolysis reaction is preferably in the range of 5 minutes to 200 hours, more preferably 30 minutes to 100 hours and most preferably 2 hours to 50 hours.

In continuous processes, the residence time is preferably 5 minutes to 100 hours, more preferably 30 minutes to 50 hours and most preferably 2 hours to 10 hours.

The reaction can be performed, for example, in a fixed bed reactor or in a suspension reactor.

The reaction mixture thus obtained may generally comprise, in addition to the desired carboxamide, further constituents, especially unconverted cyanohydrin and any acetone and/or acetaldehyde used. Accordingly, the reaction mixture can be purified, in the course of which, for example, unconverted cyanohydrin can be cleaved to acetone and hydrocyanic acid in order to use them again to prepare the cyanohydrin. The same applies to the acetone and/or acetaldehyde removed.

In addition, the reaction mixture comprising the purified carboxamide can be purified to remove further constituents by means of ion exchange columns.

For this purpose, cation exchangers and anion exchangers in particular can be used. Ion exchangers suitable for this purpose are known per se. For example, suitable cation exchangers can be obtained by sulphonating styrene-divinylbenzene copolymers. Basic anion exchangers include quaternary ammonium groups which are bonded covalently to styrene-divinylbenzene copolymers.

The purification of α-hydroxycarboxamides is described in detail, inter alia, in EP-A-0686623.

The cyanohydrin used for the hydrolysis can be obtained in any way. In the process according to the invention, the purity of the carbonitrile, for example of the cyanohydrin, is generally uncritical. Accordingly, purified or unpurified carbonitrile can be used for the hydrolysis reaction. For example, it is possible to react, for example, a ketone, especially acetone, or an aldehyde, for example acetaldehyde, propanal, butanol, with hydrocyanic acid to give the corresponding cyanohydrin. Particular preference is given in this context to converting acetone and/or acetaldehyde in a typical manner using a small amount of alkali or of an amine as the catalyst.

The above-described hydrolysis reaction serves as an intermediate step in processes for preparing alkyl(meth)acrylates. Processes which may have a hydrolysis step of cyanohydrins for the preparation of (meth)acrylic acid and/or alkyl(meth)acrylates are detailed, inter alia, in EP-A-0 406 676, EP-A-0 407 811, EP-A-0 686 623 and EP-A-0 941 984.

Proceeding from cyanohydrins, the resulting α-hydroxycarboxamide can be converted, for example, to (meth)acrylamide which can then be converted with an alkyl formate, for example methyl formate, or an alcohol to alkyl(meth)acrylate, especially methyl methacrylate. The reaction steps detailed above are described in detail in EP-A-0 406 676 and EP-A-0 686 623.

The preparation of alkyl(meth)acrylates proceeding from cyanohydrins can also be effected via a dehydration of alkyl α-hydroxycarboxylates which have been obtained beforehand from α-hydroxycarboxamides by alcoholysis or transesterification. The individual steps of this reaction path are described in detail, for example, in EP-A-0 407 811 or EP-A-0 941 984.

In a particularly preferred embodiment, alkyl(meth)acrylates can be obtained in a simple and inexpensive manner from carbonyl compounds, hydrocyanic acid and alcohols by processes which comprise the following steps:
A) formation of at least one cyanohydrin by reacting at least one carbonyl compound with hydrocyanic acid;
B) hydrolysis of the cyanohydrin or of the cyanohydrins to form at least one α-hydroxycarboxamide;
C) alcoholysis of the α-hydroxycarboxamide or of the α-hydroxycarboxamides to obtain at least one alkyl α-hydroxycarboxylate;
D) transesterifying the alkyl α-hydroxycarboxylate or alkyl α-hydroxycarboxylates with (meth)acrylic acid to form at least one alkyl(meth)acrylate and at least one α-hydroxycarboxylic acid;
E) dehydrating the α-hydroxycarboxylic acid or the α-hydroxycarboxylic acids to form (meth)acrylic acid.

Processes which comprise a step D) in particular can achieve particular advantages. For instance, processes which have a transesterification step can afford alkyl(meth)acrylates in high yields. This is especially true in comparison with the processes described in EP-A-0941984, in which the alkyl α-hydroxycarboxylates are dehydrated directly to the alkyl(meth)acrylates. Surprisingly, it was found that the additional reaction step of the transesterification of the alkyl α-hydroxycarboxylate with (meth)acrylic acid allows higher selectivities to be achieved overall. The formation of by-products is unusually low here. Moreover, especially taking account of the high selectivity, high conversions are achieved. The process according to the invention can be performed inexpensively, especially with low energy demand. The catalysts used here for the dehydration and transesterification can be used over a long period without the selectivity or the activity decreasing.

Steps A) and B) have been described in detail above. In the next step C), the α-hydroxycarboxamide thus obtained can be converted to the alkyl α-hydroxycarboxylate. This can be done, for example, by the use of alkyl formates. Especially suitable is methyl formate or a mixture of methanol and carbon monoxide, this reaction being described by way of example in EP-A-0407811.

Preference is given to converting the α-hydroxycarboxamide by alcoholysis with an alcohol which comprises preferably 1-10 carbon atoms, more preferably 1 to 5 carbon atoms. Preferred alcohols include methanol, ethanol, propanol, butanol, especially n-butanol and 2-methyl-1-propanol, pentanol, hexanol, heptanol, 2-ethylhexanol, octanol, nonanol and decanol. The alcohol used is more preferably methanol and/or ethanol, very particular preference being given to methanol. The reaction of carboxamides with alcohols to obtain carboxylic esters is common knowledge.

This reaction can be accelerated, for example, by basic catalysts. These include homogeneous catalysts and heterogeneous catalysts.

The homogeneous catalysts include alkali metal alkoxides and organometallic compounds of titanium, tin and aluminium. Preference is given to using a titanium alkoxide or tin alkoxide, for example titanium tetraisopropoxide or tin tetrabutoxide. The heterogeneous catalysts include magnesium oxide, calcium oxide and basic ion exchangers as have been described above.

The molar ratio of α-hydroxycarboxamide to alcohol, for example α-hydroxyisobutyramide to methanol, is not critical per se, and is preferably in the range of 2:1-1:20.

The reaction temperature may likewise be within wide ranges, the reaction rate increasing with increasing temperature. The upper temperature limit arises generally from the boiling point of the alcohol used. The reaction temperature is preferably in the range of 40-300° C., more preferably 160-240° C. The reaction may, depending on the reaction temperature, be performed at reduced or elevated pressure. This reaction is performed preferably in a pressure range of 0.5-35 bar, more preferably 5 to 30 bar.

Typically, the ammonia formed is discharged from the reaction system, the reaction in many cases being performed at the boiling point.

The ammonia released in the alcoholysis can be recycled to the overall process easily. For example, ammonia can be reacted with methanol to give hydrocyanic acid. This is detailed, for example, in EP-A-0941984. In addition, hydrocyanic acid can be obtained from ammonia and methane by the BMA or Andrussow process, these processes being described in Ullmann's Encyclopedia of Industrial Chemistry, 5th edition on CD-ROM, under “inorganic cyano compounds”.

In a next step D), the alkyl α-hydroxycarboxylate is reacted with (meth)acrylic acid to obtain alkyl(meth)acrylate and α-hydroxycarboxylic acid.

In a further aspect of the present invention, alkyl α-hydroxycarboxylates can be reacted with (meth)acrylic acid. The (meth)acrylic acids usable for this purpose are known per se and can be obtained commercially. In addition to acrylic acid (propenoic acid) and methacrylic acid (2-methylpropenoic acid), these include in particular derivatives which comprise substituents. The suitable substituents include halogens such as chlorine, fluorine and bromine, and alkyl groups which may comprise preferably 1 to 10, more preferably 1 to 4 carbon atoms. These include β-methylacrylic acid (butenoic acid), α,β-dimethylacrylic acid, β-ethylacrylic acid and β,β-dimethylacrylic acid. Preference is given to acrylic acid (propenoic acid) and methacrylic acid (2-methylpropenoic acid), particular preference being given to methacrylic acid.

The alkyl α-hydroxycarboxylates used for this purpose are known per se, the alcohol radical of the ester comprising preferably 1 to 20 carbon atoms, in particular 1 to 10 carbon atoms and more preferably 1 to 5 carbon atoms. Preferred alcohol radicals derive from methanol, ethanol, propanol, butanol, especially n-butanol and 2-methyl-1-propanol, pentanol, hexanol and 2-ethylhexanol, particular preference being given to methanol and ethanol.

The acid radical of the alkyl α-hydroxycarboxylates used for the transesterification derives preferably from the (meth)acrylic acid which can be obtained by dehydrating the α-hydroxycarboxylic acid. When, for example, methacrylic acid is used, α-hydroxyisobutyric ester is used. When, for example, acrylic acid is used, preference is given to using α-hydroxyisopropionic acid.

Alkyl α-hydroxycarboxylates used with preference are methyl α-hydroxypropionate, ethyl α-hydroxypropionate, methyl α-hydroxyisobutyrate and ethyl α-hydroxyisobutyrate.

As well as the reactants, the reaction mixture may comprise further constituents, for example solvents, catalysts, polymerization inhibitors and water.

The reaction of the alkylhydroxycarboxylic ester with (meth)acrylic acid can be catalysed by at least one acid or at least one base. In this context, it is possible to use either homogeneous or heterogeneous catalysts. Particularly suitable acidic catalysts are in particular inorganic acids, for example sulphuric acid or hydrochloric acid, and organic acids, for example sulphonic acids, in particular p-toluenesulphonic acid, and acidic cation exchangers.

The particularly suitable cation exchange resins include in particular sulphonic acid-containing styrene-divinylbenzene polymers. Particularly suitable cation exchange resins can be obtained commercially from Rohm&Haas under the trade name Amberlyst® and from Bayer under the trade name Lewatit®.

The concentration of catalyst is preferably in the range of 1 to 30% by weight, more preferably 5 to 15% by weight, based on the sum of the alkyl α-hydroxycarboxylate used and of the (meth)acrylic acid used.

The polymerization inhibitors usable with preference include phenothiazine, tert-butylcatechol, hydroquinone monomethyl ether, hydroquinone, 4-hydroxy-2,2,6,6-tetramethylpiperidinooxyl (TEMPOL) or mixtures thereof; the effectiveness of these inhibitors can be improved in some cases by use of oxygen. The polymerization inhibitors may be used in a concentration in the range of 0.001 to 2.0% by weight, more preferably in the range of 0.01 to 0.2% by weight, based on the sum of the alkyl α-hydroxycarboxylate used and of the (meth)acrylic acid used.

The reaction is performed preferably at temperatures in the range of 50° C. to 200° C., more preferably 70° C. to 130° C., in particular 80° C. to 120° C. and most preferably 90° C. to 110° C.

The reaction may be performed at reduced or elevated pressure depending on the reaction temperature. Preference is given to performing this reaction in the pressure range of 0.02-5 bar, in particular 0.2 to 3 bar and more preferably 0.3 to 0.5 bar.

The molar ratio of (meth)acrylic acid to the alkyl α-hydroxycarboxylate is preferably in the range of 4:1-1:4, in particular 3:1 to 1:3, and more preferably in the range of 2:1-1:2.

The selectivity is preferably at least 90%, more preferably 98%. The selectivity is defined as the ratio of the sum of the amounts of alkyl(meth)acrylates and α-hydroxycarboxylic acids formed based on the sum of the amounts of alkyl α-hydroxycarboxylate and (meth)acrylic acid converted.

In a particular aspect of the present invention, the transesterification can be effected in the presence of water. The water content is preferably in the range of 0.1-50% by weight, more preferably 0.5-20% by weight and most preferably 1-10% by weight, based on the weight of the alkyl α-hydroxycarboxylate used.

The addition of small amounts of water surprisingly allows the selectivity of the reaction to be increased. In spite of water addition, the formation of methanol can be kept surprisingly low. At a water concentration of 10 to 15% by weight, based on the weight of the alkyl α-hydroxycarboxylate used, preferably less than 5% by weight of methanol forms at a reaction temperature of 120° C. and a reaction time or residence time of 5 to 180 min.

The transesterification can be performed batchwise or continuously, preference being given to continuous processes.

The reaction time of the transesterification depends upon the molar masses used and the reaction temperature, and this parameter may be within wide ranges. The reaction time of the transesterification of the alkyl α-hydroxycarboxylate with (meth)acrylic acid is preferably in the range of 30 seconds to 15 hours, more preferably 5 minutes to 5 hours and most preferably 15 minutes to 3 hours.

In continuous processes, the residence time is preferably 30 seconds to 15 hours, more preferably 5 minutes to 5 hours and most preferably 15 minutes to 3 hours.

In the preparation of methyl methacrylate from methyl α-hydroxyisobutyrate, the temperature is preferably 60 to 130° C., more preferably 80 to 120° C. and most preferably 90 to 110° C. The pressure is preferably in the range of 50 to 1000 mbar, more preferably 300 to 800 mbar. The molar ratio of methacrylic acid to methyl α-hydroxyisobutyrate is preferably in the range of 2:1-1:2, in particular 1.5:1-1:1.5.

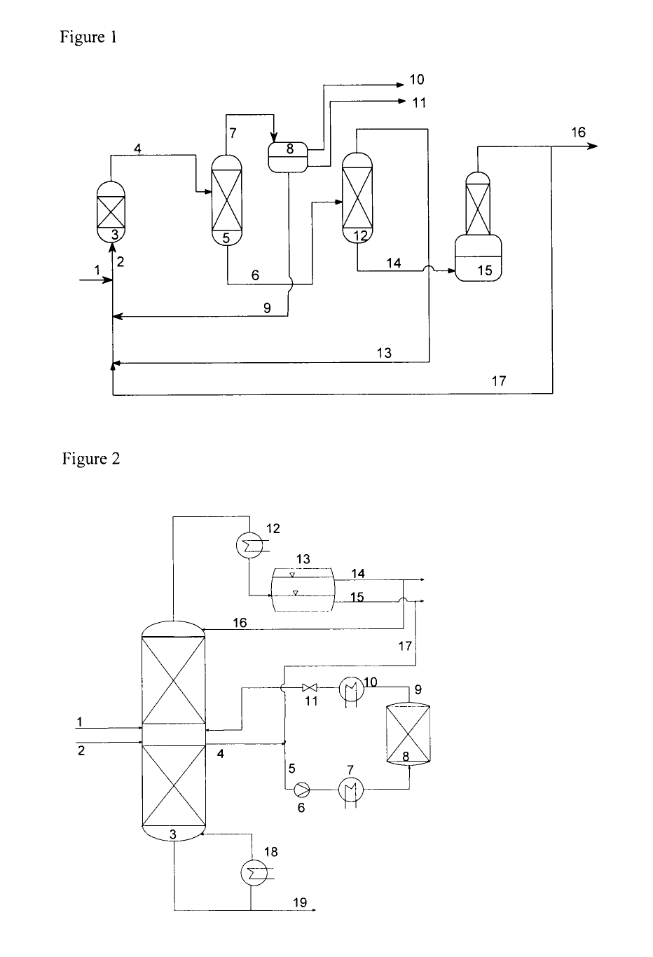

For example, the transesterification can be effected in the plant detailed in FIG. 1. The hydroxycarboxylic ester, for example methyl hydroxyisobutyrate, is fed via line (1) to a fixed bed reactor (3) which comprises a cation exchange resin. (Meth)acrylic acid, for example 2-methylpropenoic acid, is added to the fixed bed reactor (3) via line (2) or line (17). Line (2) may be connected to further lines, for example line (9) and line (13), in order thus to reduce the number of feed lines into the reactor. However, lines (9), (13) and/or (17) may also lead directly into the fixed bed reactor. Under the aforementioned reaction conditions, a reaction mixture is formed which comprises, as well as methanol and unconverted methyl hydroxyisobutyrate and methacrylic acid, the hydroxyisobutyric acid and methyl methacrylate reaction products. This reaction mixture is passed via line (4) into a still (5). In the still (5), water, methyl methacrylate and methanol are obtained as the distillate, which is fed via line (7) as the top product to a phase separator (8). Methacrylic acid and methanol collect in the upper phase, which is withdrawn from the system via line (10). Principally water collects in the lower phase of the phase separator (8) and is removed from the system via line (11) or can be fed to the fixed bed reactor (3) via line (9).

Methyl hydroxyisobutyrate, hydroxyisobutyric acid and methacrylic acid can be obtained from the bottoms, and can be passed via line (6) into a second still (12). In this still, methyl hydroxyisobutyrate and methacrylic acid are distilled off and are recycled via line (13) to the transesterification. The hydroxyisobutyric acid present in the distillation bottoms is passed via line (14) into a reactor for dehydration (15). The methacrylic acid obtained in this way can be fed via line (17) to the transesterification detailed above or withdrawn from the system via line (16).

In a particularly preferred embodiment, the transesterification can be effected in a still. In this case, the catalyst can be added in any region of the still. For example, the catalyst can be provided in the region of the bottom or in the region of the column. However, the reactants here should be brought into contact with the catalyst. In addition, catalyst can be provided in a separate region of the still, in which case this region is connected to the further regions of the still, for example the bottom and/or the column. This separate arrangement of the catalyst region is preferred.

This preferred embodiment surprisingly succeeds in increasing the selectivity of the reaction. In this context, it should be emphasized that the pressure of the reaction can be adjusted independently of the pressure within the distillation columns. This allows the boiling temperature to be kept low without the reaction time and the residence time rising correspondingly. In addition, the temperature of the reaction can be varied over a wide range. This allows the reaction time to be shortened. In addition, the volume of catalyst can be selected as desired without any need to take account of the geometry of the column. In addition, it is possible, for example, to add a further reactant. All of these measures can contribute to an increase in the selectivity and the productivity, surprising synergistic effects being achieved.

The alkyl α-hydroxycarboxylate, for example methyl α-hydroxyisobutyrate, is fed to the still. In addition, (meth)acrylic acid, for example methacrylic acid, is introduced into the still. The distillation conditions are preferably configured such that exactly one product is discharged from the still by distillation, the second product remaining in the bottoms and being removed continuously therefrom. When alcohols having a low carbon number are used, especially ethanol or methanol, preference is given to withdrawing the alkyl(meth)acrylate from the reaction mixture by distillation. The reactants are passed cyclically through the catalyst region. This continuously forms alkyl(meth)acrylate and α-hydroxycarboxylic acid.

A preferred embodiment of the reactive distillation is shown schematically in FIG. 2. The reactants may be introduced into the distillation column (3) via one common line (1) or separately via two lines (1) and (2). The reactants are preferably added via separate lines. The reactants can be fed at the same stage or in any position in the column.

The temperature of the reactants can be adjusted by means of a heat exchanger in the feed, the units needed for this purpose not being shown in FIG. 1. In a preferred variant, the reactants are metered separately into the column, the lower-boiling components being metered in below the position for the feeding of the higher-boiling compounds. In this case, the lower-boiling component is preferably added in vaporous form.

For the present invention, any multistage distillation column (3) which has two or more separating stages may be used. The number of separating stages used in the present invention is the number of trays in a tray column or the number of theoretical plates in the case of a column with structured packing or a column with random packings.

Examples of a multistage distillation column with trays include those such as bubble-cap trays, sieve trays, tunnel-cap trays, valve trays, slot trays, slotted sieve trays, bubble-cap sieve trays, jet trays, centrifugal trays; for a multistage distillation column with random packings, those such as Raschig rings, Lessing rings, Pall rings, Berl saddles, Intalox saddles; and, for a multistage distillation column with structured packings, those such as Mellapak (Sulzer), Rombopak (Kühni), Montz-Pak (Montz) and structured packings with catalyst pockets, for example Kata-Pak.

A distillation column with combinations of regions of trays, of regions of random packings or of regions of structured packings may likewise be used.

The column (3) may be equipped with internals. The column preferably has a condenser (12) for condensing the vapour and a bottom evaporator (18).

The distillation apparatus preferably has at least one region, known hereinafter as reactor, in which at least one catalyst is provided. This reactor may be within the distillation column. However, this reactor is preferably arranged outside the column (3) in a separate region, one of these preferred embodiments being explained in detail in FIG. 2.

In order to carry out the transesterification reaction in a separate reactor (8), it is possible within the column to collect a portion of the liquid phase flowing downwards by means of a collector and to pass it out of the column as a substream (4). The position of the collector is determined by the concentration profile in the column of the individual components. The concentration profile can be regulated by means of the temperature and/or the reflux. The collector is preferably positioned such that the stream conducted out of the column contains both reactants, more preferably the reactants in sufficiently high concentration and most preferably in a molar acid:ester ratio=1.5:1 to 1:1.5. In addition, a plurality of collectors may be provided at various points in the distillation column, in which case the amount of reactants withdrawn can be used to adjust the molar ratios.

It is additionally possible for a further reactant, for example water, to be metered into the stream conducted out of the column, in order to adjust the acid/ester product ratio in the cross-transesterification reaction or to increase the selectivity. The water can be fed from outside via a line (not shown in FIG. 1) or from a phase separator (13). The pressure of the stream (5) enriched with water can then be increased by a means for pressure increase (6), for example a pump.

An increase in the pressure can reduce or prevent formation of steam in the reactor, for example a fixed bed reactor. This allows uniform flow-through of the reactor and wetting of the catalyst particles. The stream can be conducted through a heat exchanger (7) and the reaction temperature adjusted. The stream can be heated or cooled as required. It is additionally possible to adjust the ester to acid product ratio via the reaction temperature.

The transesterification reaction takes place over the catalyst in the fixed bed reactor (8). The flow through the reactor may be downwards or upwards. The reactor output stream (9) comprising the products and the unconverted reactants to a certain degree, the content of the components in the reactor waste stream depending upon the residence time, the catalyst mass, the reaction temperature and the reactant ratio and the amount of water added, is first passed through a heat exchanger (10) and adjusted to a temperature which is advantageous for the introduction into the distillation column. Preference is given to setting the temperature which corresponds to the temperature in the distillation column at the point of introduction of the stream.

The position where the stream leaving the reactor is returned into the column may lie above or below the position for the withdrawal of the reactor feed, but will preferably be above it. Before the recycling into the column, the stream may be decompressed through a valve (11), which preferably establishes the same pressure level as in the column. In this context, the distillation column preferably has a lower pressure. This configuration offers the advantage that the boiling points of the components to be separated are lower, as a result of which the distillation can be carried out at a lower temperature level, as a result of which it saves energy and is more thermally gentle.

In the distillation column (3), the product mixture is then separated. The low boiler, preferably the ester formed in the transesterification, is removed via the top. The distillation column is preferably operated such that the water added upstream of the fixed bed reactor is likewise removed as the top product. The vaporous stream drawn off at the top is condensed in a condenser (12) and then separated in a decanter (13) into the aqueous phase and product ester-containing phase. The aqueous phase can be discharged to the workup via a line (15) or returned fully or partly back into the reaction via line (17) as a stream. The stream of the ester-containing phase can be conducted via line (14) partly as reflux (16) to the column or discharged partly from the still. The high boiler, preferably the acid formed in the cross-transesterification, is discharged from the column (19) as a bottom stream.

This preferred embodiment surprisingly succeeds in increasing the selectivity of the reaction. In this context, it should be emphasized that the pressure of the reaction can be adjusted independently of the pressure within the distillation columns. This allows the boiling temperature to be kept low without the reaction time and the residence time rising correspondingly. In addition, the temperature of the reaction can be varied over a wide range. This allows the reaction time to be shortened. In addition, the volume of catalyst can be selected as desired without any need to take account of the geometry of the column. In addition, it is possible, for example, to add a further reactant.

The α-hydroxycarboxylic acid obtained from the reaction, for example hydroisobutyric acid, can be dehydrated in a known manner in a further step E). In general, the α-hydroxycarboxylic acid, for example the α-hydroxyisobutyric acid, is heated in the presence of a metal salt, for example of alkali metal and/or alkaline earth metal salts, to temperatures in the range of 160-300° C., more preferably in the range of 200 to 240° C., generally to obtain the (meth)acrylic acid and water. The suitable metal salts include sodium hydroxide, potassium hydroxide, calcium hydroxide, barium hydroxide, magnesium hydroxide, sodium sulphite, sodium carbonate, potassium carbonate, strontium carbonate, magnesium carbonate, sodium bicarbonate, sodium acetate, potassium acetate and sodium dihydrogenphosphate.

The dehydration of the α-hydroxycarboxylic acid can be performed preferably at a pressure in the range of 0.05 bar to 2.5 bar, more preferably in the range of 0.1 bar to 1 bar.

The dehydration of α-hydroxycarboxylic acids is described, for example, in DE-A-176 82 53.

The (meth)acrylic acid thus obtained can in turn be used to prepare alkyl(meth)acrylates. In addition, (meth)acrylic acid is a commercial product. Surprisingly, the process for preparing alkyl(meth)acrylates can accordingly likewise serve to prepare (meth)acrylic acid, in which case the product ratio of alkyl(meth)acrylates to (meth)acrylic acid can be regulated easily by the concentration of water in the transesterification of the alkyl α-hydroxycarboxylate and/or by the reaction temperature.

The present invention will be illustrated in detail hereinafter with reference to examples.

### EXAMPLE 1

### Culturing Conditions

The precultures were grown in a volume of 5 ml in glass tubes, shaking at 30° C. over the course of 24 h. 100 ml of the main culture were inoculated with 1 ml of the preculture and shaken in an Erlenmeyer flask with a total volume of 1000 ml at 25° C. for 42 h.

Medium for the preculture (pH 7.0)

| | | |
|---|---|---|
| K₂HPO₄ | 7 | g |
| KH₂PO₄ | 3 | g |
| sodium citrate | 0.5 | g |
| glycerol | 2 | g |
| FeSO₄•7 H₂O | 0.004 | g |
| MgSO₄•7 H₂O | 0.1 | g |
| acetamide | 2 | g |
| trace salt solution | 0.1 | ml |
| demineralized water | Ad. 1000 | ml |

Medium for the main culture (pH 7.0)

| | | |
|---|---|---|
| K₂HPO₄ | 7 | g |
| KH₂PO₄ | 3 | g |
| sodium citrate | 0.5 | g |
| glycerol | 2 | g |
| FeSO₄•7 H₂O | 0.004 | g |
| MgSO₄•7 H₂O | 0.1 | g |
| acetamide | 10 | g |
| trace salt solution | 0.1 | ml |
| demineralized water | Ad. 1000 | ml |

Trace salt solution

| | | |
|---|---|---|
| EDTA, Na₂•2 H₂O | 158 | mg |
| Na₂MoO₄•2 H₂O | 4.7 | mg |
| ZnSO₄•7 H₂O | 70 | mg |
| MnSO₄•4 H₂O | 18 | mg |
| FeSO₄•7 H₂O | 16 | mg |
| CuSO₄•5 H₂O | 4.7 | mg |
| CoSO₄•6 H₂O | 5.2 | mg |
| demineralized water | Ad. 1000 | ml |

### EXAMPLE 2

### Isolation and Identification of the Microorganisms

The two strains MA32 and MA113 were selected by determining the nitrile hydratase activity of the resting cells in the presence of 2 mM potassium cyanide.

Properties of MA32:

| | |
|---|---|
| Cell form | rods |
| Width | 0.6-0.8 μm |
| Length | 1.5-3.0 μm |
| Motility | + |
| Flagella | polar >1 |
| Gram reaction | − |
| Lysis by 3% KOH | + |
| Aminopeptidase (Cerny) | + |
| Oxidase | + |
| Catalase | + |
| Growth at 41° C. | − |
| Substrate utilization | |
| adipate | − |
| citrate | + |
| malate | + |
| phenylacetate | − |
| D-glucose | + |
| maltose | − |
| mannitol | + |
| arabinose | + |
| mannose | + |
| trehalose | + |
| sorbitol | + |
| erythrol | + |
| citraconate | + |
| inositol | + |
| ADH | + |
| Urease | − |
| Hydrolysis of gelatin | + |
| Hydrolysis of esculin | + |
| Levan from sucrose | + |
| Denitrification | + |
| Lecithinase | + |
| Fluorescence | + |
| Pyocyanin | − |

The profile of the cellular fatty acids is typical of Group I Pseudomonas.

Analysis of a 484 bp-long segment of the 16S rRNA revealed a 100% agreement with the sequence of Pseudomonas marginalis.

It was possible, taking account of all the data, to identify MA32 as Pseudomonas marginalis.

Properties of MA113:

| | |
|---|---|
| Cell form | rods |
| Width | 0.6-0.8 μm |
| Length | 1.5-3.0 μm |
| Motility | + |
| Flagella | polar >1 |
| Gram reaction | − |
| Lysis by 3% KOH | + |
| Aminopeptidase (Cerny) | + |
| Oxidase | + |
| Catalase | + |
| Growth at 41° C. | − |
| Substrate utilization | |
| adipate | − |
| citrate | + |
| malate | + |
| phenylacetate | + |
| D-glucose | + |
| maltose | − |
| mannitol | − |
| arabinose | − |
| mannose | − |
| trehalose | − |
| inositol | − |
| β-alanine | + |
| α-ketoglutarate | + |
| benzylamine | + |
| hippurate | + |
| azelate | + |
| D-mandelate | + |
| ADH | + |
| Urease | − |
| Hydrolysis of gelatin | − |
| Hydrolysis of esculin | − |
| Levan from sucrose | − |
| Denitrification | − |
| Lecithinase | − |
| Fluorescence | + |
| Pyocyanin | − |

The profile of the cellular fatty acids is typical of Group I Pseudomonas.

Analysis of a 476 bp-long segment of the 16S rRNA revealed a 100% agreement with the sequence of Pseudomonas putida.

It was possible, taking account of all the data, to identify MA32 as Pseudomonas putida.

### EXAMPLE 3

### Influence of Cyanide on the Activity of the Nitrile Hydratase

The cells were grown as described in Example 1, removed from the culture medium by centrifugation and resuspended in standard buffer (50 mM potassium phosphate buffer of pH 7.5). 50 μl of this cell suspension were added to 700 μl of the standard buffer which contained 0, 21.4, 53.6 and 107.1 mM potassium cyanide (final concentration 0, 20, 50, 100 mM cyanide). The reaction was started by adding 200 μl of a 200 mM solution of the nitrile in standard buffer which in each case had the same cyanide concentration as the remaining reaction solution. The concentration of the cells in the cell suspension was in this case such that the nitrile was 16% converted in the mixture without cyanide after 10 min at 20° C. After 10 min at 20° C., the reaction was stopped by adding 20 μl of semiconcentrated phosphoric acid, and the cells were removed by centrifugation.

The activity of one U is defined as the amount of enzyme which converts 1 μmol of methacrylonitrile to the amide in one minute. If the acid was also formed in addition to the amide, one U was defined as the amount of enzyme which converts 1 μmol of methacrylonitrile to the amide and acid in one minute.

The conversion was determined by HPLC analysis. For this purpose, a column with Intersil ODS-3V (GL Sciences Inc.) was used, and the mobile phase used was a mixture of 10 mM potassium phosphate buffer of pH 2.3 and acetonitrile in a ratio of 85:15. The flow rate was 1 ml/min. The detection was by means of UV at 200 nm.

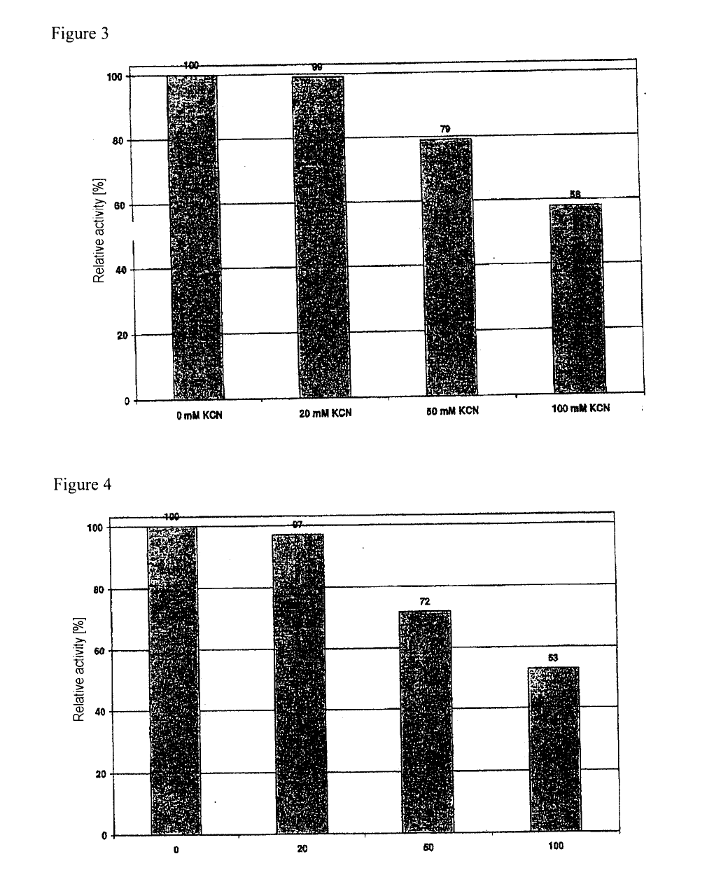

The relative activities for the conversion of methacrylonitrile as a function of the cyanide concentration are shown in FIG. 3 and in FIG. 4.

### EXAMPLE 4

### Conversion of Acetone Cyanohydrin with Resting Cells of Pseudomonas marginalis MA32 and Pseudomonas putida MA 13

Pseudomonas marginalis MA32 and Pseudomonas putida MA113 cells were grown and centrifuged as described in Example 1. An amount of the cells which contained 1.16 g of dry biomass was diluted with 50 mM potassium phosphate buffer of pH 8.0 to a final volume of 50 ml. In addition, 0.02 mM 2-methyl-1-propaneboronic acid was added to the reaction mixture. Freshly distilled acetone cyanohydrin was added continuously at 4° C. with vigorous stirring at such a rate that the concentration did not exceed 5 g/l at any point during the reaction. The pH was kept constant at 7.5. The reaction was monitored by HPLC as described in Example 3. After 140 min, 10.0 g of the nitrile had been completely converted to 10.7 g of amide and 1.4 g of acid.

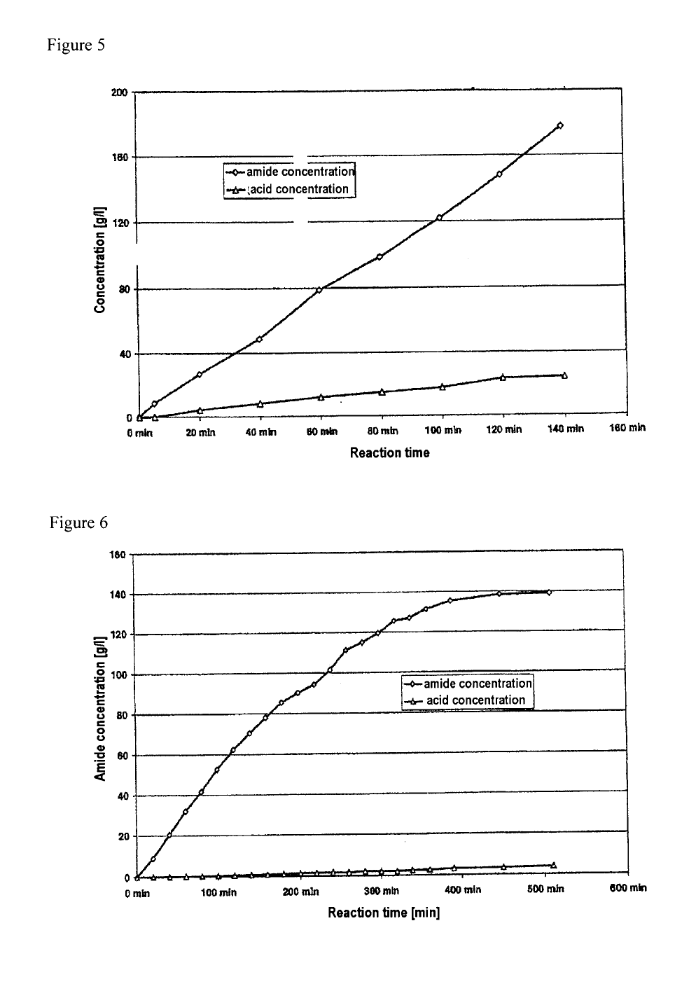

The reaction profile against time achieved with the strains MA113 and MA31 is shown in FIG. 5 and FIG. 6.

### EXAMPLE 5

In a reactive still shown in FIG. 2, 4619 g of methyl α-hydroxyisobutyrate (MHIB) and 3516 g of methacrylic acid (MAA) were supplied over a period of 48 hours. The reaction was performed at a temperature of 120° C. and a pressure of 250 mbar. α-Hydroxyisobutyric acid formed was removed from the bottom. Methyl methacrylate (MMA) was distilled off. The reaction was performed in the presence of 16% by weight of water based on the weight of methyl α-hydroxyisobutyrate. The reaction was performed using an acidic catalyst (cation exchanger; Lewatit® K2431 from Bayer).

The selectivity, defined as the ratio of amounts of methyl methacrylate (MMA) and α-hydroxyisobutyric acid (HIBA) formed to amounts of MHIB and MMA converted, was 99%.

The α-hydroxyisobutyric acid obtained from the process was dehydrated according to DE-A 17 68 253.

The overall selectivity is 98.5%, which is defined as the ratio of amount of MMA formed to the amount of MHIB converted.

### EXAMPLE 6

Methyl methacrylate was prepared by dehydrating methyl α-hydroxyisobutyrate. This reaction was performed according to EP-A-0941984. A mixture of 20 g of sodium dihydrogenphosphate and 80 g of water was added to 60 g of silica gel. The water was removed from the mixture under a reduced pressure. The residue was dried at 150° C. overnight in order to obtain a catalyst. 10 g of the catalyst obtained were introduced into a quartz tube which had been equipped with an evaporator. The quartz tube was heated with an oven, and the temperature of the catalyst layer was about 400° C. A mixture of methanol and methyl α-hydroxyisobutyrate (2:1) was continuously evaporated at a rate of 10 g per hour and passed over the catalyst layer. The selectivity of the reaction, defined as the ratio of amount of MMA formed to the amount of MHIB converted, was 88%.

### EXAMPLES 7 TO 23

Example 1 was essentially repeated, except that no water was added to the reaction mixture. The reaction was effected under the conditions specified in Table 1, more particularly with regard to the temperature, residence time and molar ratio of the reactants. The selectivity of the reactions, defined as the ratio of amounts of MMA and HIBA formed to amounts of MHIB and MAA converted, is likewise shown in Table 1.

TABLE 1

| | Reaction | Molar | | |
|---|---|---|---|---|
| | temperature | MHIB/MAA | Residence time | Selectivity |
| Example | [° C.] | ratio | [min] | [%] |
| 7 | 120 | 1.00 | 28.33 | 93.21 |
| 8 | 90 | 1.00 | 42.50 | 95.06 |
| 9 | 100 | 1.00 | 42.50 | 94.81 |
| 10 | 110 | 1.00 | 42.50 | 94.64 |
| 11 | 120 | 1.00 | 42.50 | 90.67 |
| 12 | 90 | 1.00 | 85.00 | 95.53 |
| 13 | 100 | 1.00 | 85.00 | 94.95 |
| 14 | 110 | 1.00 | 85.00 | 93.55 |
| 15 | 120 | 1.00 | 85.00 | 91.78 |
| 16 | 90 | 1.00 | 170.00 | 94.83 |
| 17 | 100 | 1.00 | 170.00 | 94.06 |
| 18 | 90 | 2.0 | 42.50 | 91.61 |
| 19 | 100 | 2.0 | 42.50 | 91.73 |
| 20 | 90 | 2.0 | 85.00 | 90.63 |
| 21 | 100 | 2.0 | 85.00 | 90.30 |
| 22 | 120 | 0.50 | 28.33 | 92.05 |
| 23 | 120 | 0.50 | 42.50 | 92.62 |

1: A process for preparing alkyl(meth)acrylates wherein the process has a step in which a cyanohydrin is hydrolysed with an enzyme whose residual activity after the conversion of methacrylonitrile in the presence of 20 mM cyanide ions at 20° C. after 30 min is at least 90% of the residual activity of the enzyme which has been used under otherwise identical conditions in the absence of cyanide ions.

2: The process according to claim 1 wherein the residual activity after the conversion in the presence of 50 mM cyanide ions is at least 60%.

3: The process according to claim 1 wherein microorganisms, or the lysate thereof, which produce and comprise the enzyme are used.

4: The process according to claim 3 wherein resting cells of the microorganism are used.

5: The process according to claim 1 wherein the purified enzyme is used.

6: The process according to claim 1 wherein the enzyme stems from microorganisms of the Pseudomonas genus.

7: The process according to claim 6 wherein the enzyme stems from microorganisms of the Pseudomonas genus deposited under number DSM 16275 and DSM 16276.

8: The process according to claim 1 wherein the hydrolysis of the cyanohydrin is performed in the presence of hydrocyanic acid or a salt of hydrocyanic acid.

9: The process according to claim 8 wherein the hydrolysis of the cyanohydrin is performed in the presence of a starting concentration of more than 0.5 mol % of cyanide to 3 mol % of cyanide, based on the cyanohydrin used.

10: The process according to claim 1 wherein the cyanohydrin is 2-hydroxy-2-methylpropionitrile or 2-hydroxypropionitrile.

11: The process according to claim 1 wherein the hydrolysis reaction is performed in the presence of a carbonyl compound.

12: The process according to claim 11 wherein the concentration of the carbonyl compound is in the range of 0.1 to 6 mol per mole of cyanohydrin.

13: The process according to claim 1 wherein the concentration of the cyanohydrin is in the range of 0.02 to 10 w/w % based on the amount of the biocatalyst as a dried cell mass.

14: The process according to claim 1 wherein the hydrolysis reaction is performed at a temperature in the range of 5 to 50° C.

15: The process according to claim 1 wherein the hydrolysis reaction is performed at a pressure in the range of 0.1 bar to 10 bar.

16: The process according to claim 1 wherein the cyanohydrin is obtained by reacting a ketone or aldehyde with hydrocyanic acid in the presence of a basic catalyst.

17: The process according to claim 1 wherein the process comprises
A) formation of at least one cyanohydrin by reacting at least one carbonyl compound with hydrocyanic acid;
B) hydrolysis of the cyanohydrin or of the cyanohydrins to form at least one a hydroxycarboxamide;
C) alcoholysis of the α-hydroxycarboxamide or of the α hydroxycarboxamides to obtain at least one alkyl α hydroxycarboxylate;
D) transesterifying the alkyl α hydroxycarboxylate or alkyl α hydroxycarboxylates with (meth)acrylic acid to form at least one alkyl(meth)acrylate and at least one α hydroxycarboxylic acid; and
E) dehydrating the α hydroxycarboxylic acid or the α hydroxycarboxylic acids to form (meth)acrylic acid.

18: The process according to claim 1 wherein methyl methacrylate is prepared.
